(19) United States
(12) Patent Application Publication
(10) Pub. No.: US 2024/0374219 A1
(43) Pub. Date: Nov. 14, 2024

(54) BODY ACTION DETECTION, IDENTIFICATION AND/OR CHARACTERIZATION USING A MACHINE LEARNING MODEL

(71) Applicant: SIVA HEALTH AG, Zurich (CH)

(72) Inventors: Sadegh Seyed MOHAMMADI, Leverkusen (DE); Steffen VOGLER, Leverkusen (DE); Matthias LENGA, Leverkusen (DE); Daniel RECHSTEINER, Leverkusen (DE); Mitja ALGE, Zurich (CH); Alexander DUSCHAU-WICKE, Zurich (CH)

(73) Assignee: SIVA HEALTH AG, Zurich (CH)

(21) Appl. No.: 18/692,060

(22) PCT Filed: Sep. 8, 2022
(86) PCT No.: PCT/EP2022/074928

§ 371 Date: Mar. 14, 2024

(30) Foreign Application Priority Data
Sep. 15, 2021 (EP) 21196763.3
Sep. 29, 2021 (EP) 21199826.5

(51) Int. Cl. A61B 5/00; A61B 5/01; A61B 5/0205; A61B 5/021; A61B 5/024; A61B 5/08; A61B 5/085; A61B 5/28; A61B 5/296

(52) U.S. Cl. CPC A61B 5/7267; A61B 5/02055; A61B 5/0823; A61B 5/7257; A61B 5/7282; A61B 5/01; A61B 5/021; A61B 5/024; A61B 5/085; A61B 5/28; A61B 5/296; A61B 2562/0204; A61B 2562/0219; A61B 2562/0271

(57) ABSTRACT

Systems, methods, and computer programs disclosed herein relate to training and using a machine learning model to detect, identify and/or characterize body actions, in particular cough events.

### FIELD

Systems, methods, and computer programs disclosed herein relate to training and using a machine learning model to detect, identify and/or characterize body actions, in particular cough events.

### BACKGROUND

Body actions are measurable events that can provide information about a person's health status. Examples of body actions include cough, snoring, sneezing, hiccups, vomiting, shouting, swallowing, wheezing, shortness of breath, chewing, teeth grinding, chills, convulsions, spasm and/or the like.

Cough is a symptom that has been experienced by every human and it is an essential protective and defensive act whose action secures the removal of mucus, noxious substances and infections from the larynx, trachea and larger bronchi. There are different “types” of coughs that can be traced back to different physiological mechanisms and/or pathological causes (K. Chung et al.: Semantics and types of cough, Pulm Pharmacol Ther. 2009, 22(2), 139-142.).

There are numerous publications dealing with the analysis of body actions and the diagnosis of diseases based on the analysis results (see e.g. N. M. Manshouri: Identifying COVID-19 by using spectral analysis of cough recordings: a distinctive classification study, Cogn Neurodyn (2021), https://doi.org/10.1007/s11571-021-09695-w; R. X. A. Pramono et al.: A Cough-Based Algorithm for Automatic Diagnosis of Pertussis, PLOS ONE, DOI:10.1371/journal.pone.0162128).

In many publications the use of machine learning systems that are trained to recognize and/or classify body actions is described.

For example, US2020/015709A1 discloses a method for cough detection in background noise environments using two classifiers, a first classifier trained to detect an explosive phase of a cough sound and a second classifier trained to detect one or more post-explosive phases of the cough sound.

L. Kvapilova et al. disclose the use of smartphones and machine learning for 24 h cough monitoring (Continuous Sound Collection Using Smartphones and Machine Learning to Measure Cough, Digit Biomark, 2019, 3, 166-175).

M. Pahar et al. disclose a machine learning based COVID-19 cough classifier which can discriminate COVID-19 positive coughs from both COVID-19 negative and healthy coughs recoded on a smartphone (COVID-19 Cough Classification using Machine Learning and Global Smartphone Recordings, 2021, arXiv:2012.01926v2).

However, the reliable detection, identification and/or characterization of body actions remains a challenge. There is still a need for improvement.

### SUMMARY

The present disclosure provides an improved solution for the detection, identification and/or characterization of body actions, in particular cough events.

In a first aspect, the present disclosure provides a computer-implemented method, the method comprising the steps:
receiving one or more signals of one or more modalities, the signal(s) being related to an event,
providing a trained machine learning model, wherein the trained machine learning model comprises an output, and, for each signal, a first input, and a second input,
for each signal:
   generating a time-domain representation of the signal,
   generating a spectrogram representation of the signal,
   inputting the time-domain representation into the first input of the trained machine learning model and the spectrogram representation into the second input of the machine learning model, wherein the trained machine learning model is configured and trained to
      generate time-domain features on the basis of the time-domain representation,
      generate spectrogram features on the basis of the spectrogram representation,
      generate a joint representation on the basis of the time-domain features and the spectrogram features of all signals, and
      generate, on the basis of the joint representation, an event information, the event information indicating whether and/or to what extent the event is related to a body action,
receiving, from the trained machine learning model the event information,
outputting the event information.

In another aspect, the present disclosure provides a computer system comprising:

a processor; and
a memory storing an application program configured to perform, when executed by the processor, an operation, the operation comprising:
receiving one or more signals of one or more modalities, the signal(s) being related to an event,
providing a trained machine learning model, wherein the trained machine learning model comprises an output, and, for each signal, a first input, and a second input,
for each signal:
   generating a time-domain representation of the signal,
   generating a spectrogram representation of the signal,
   inputting the time-domain representation into the first input of the trained machine learning model and the spectrogram representation into the second input of the machine learning model, wherein the trained machine learning model is configured and trained to
      generate time-domain features on the basis of the time-domain representation,
      generate spectrogram features on the basis of the spectrogram representation,
      generate a joint representation on the basis of the time-domain features and the spectrogram features of all signals, and
      generate, on the basis of the joint representation, an event information, the event information indicating whether and/or to what extent the event is related to a body action,
receiving, from the trained machine learning model the event information,
outputting the event information.

In another aspect, the present disclosure provides a non-transitory computer readable medium having stored thereon software instructions that, when executed by a processor of a computer system, cause the computer system to execute the following steps:
receiving one or more signals of one or more modalities, the signal(s) being related to an event,
providing a trained machine learning model, wherein the trained machine learning model comprises an output, and, for each signal, a first input, and a second input,
for each signal:
   generating a time-domain representation of the signal,
   generating a spectrogram representation of the signal,
   inputting the time-domain representation into the first input of the trained machine learning model and the spectrogram representation into the second input of the machine learning model, wherein the trained machine learning model is configured and trained to
      generate time-domain features on the basis of the time-domain representation,
      generate spectrogram features on the basis of the spectrogram representation,
      generate a joint representation on the basis of the time-domain features and the spectrogram features of all signals, and
      generate, on the basis of the joint representation, an event information, the event information indicating whether and/or to what extent the event is related to a body action,
receiving, from the trained machine learning model the event information,
outputting the event information.

Further aspects of the present invention are disclosed in the independent claims, the specification, and the drawings.

### DETAILED DESCRIPTION

The invention will be more particularly elucidated below without distinguishing between the aspects of the invention (method, computer system, computer-readable storage medium). On the contrary, the following elucidations are intended to apply analogously to all the aspects of the invention, irrespective of in which context (method, computer system, computer-readable storage medium) they occur.

If steps are stated in an order in the present description or in the claims, this does not necessarily mean that the invention is restricted to the stated order. On the contrary, it is conceivable that the steps can also be executed in a different order or else in parallel to one another, unless one step builds upon another step, this absolutely requiring that the building step be executed subsequently (this being, however, clear in the individual case). The stated orders are thus preferred embodiments of the invention.

As used herein, the articles “a” and “an” are intended to include one or more items and may be used interchangeably with “one or more” and “at least one.” As used in the specification and the claims, the singular form of “a”, “an”, and “the” include plural referents, unless the context clearly dictates otherwise. Where only one item is intended, usually the term “one” or similar language is used. Also, as used herein, the terms “has”, “have”, “having”, or the like are intended to be open-ended terms. Further, the phrase “based on” is intended to mean “based at least partially on” unless explicitly stated otherwise. Further, the phrase “based on” may mean “in response to” and be indicative of a condition for automatically triggering a specified operation of an electronic device (e.g., a controller, a processor, a computing device, etc.) as appropriately referred to herein.

Some implementations of the present disclosure will be described more fully hereinafter with reference to the accompanying drawings, in which some, but not all implementations of the disclosure are shown. Indeed, various implementations of the disclosure may be embodied in many different forms and should not be construed as limited to the implementations set forth herein; rather, these example implementations are provided so that this disclosure will be thorough and complete, and will fully convey the scope of the disclosure to those skilled in the art.

The present disclosure provides means for the detection and/or characterization of body actions.

A “body action” is any action that is caused and/or produced by the body of an organism or a part thereof. The organism is preferably a human being.

A body action can be caused by an internal stimulus or it can be a reaction to an external stimulus. A body action can be an unconscious or a conscious action. A body action can be intentional or unintentional (such as a reflex).

Examples of a body action include but are not limited to cough, snoring, sneezing, hiccups, vomiting, shouting, swallowing, wheezing, shortness of breath, chewing, teeth grinding, chills, convulsions, spasm and/or the like.

In a preferred embodiment of the present disclosure, the body action is a cough event.

Body actions are “measurable” in the sense that they produce and/or are accompanied by one or more signals that can be detected by physical and/or chemical measurement methods.

A “signal” is a function that conveys information about a phenomenon and/or an event. Preferably, a signal is a change in a physical quantity over time. A signal can also be considered as a representation of a body action.

For example, coughing produces sound that can be detected as an acoustic signal. At the same time, coughing leads to movements of at least the upper body, which can be detected e.g. as a visible signal by a camera and/or as an electrical signal by means of acceleration sensors. In addition, cough produces electromyographic signals.

The way in which a signal is generated, present and/or detected is also referred to as the “modality” of the signal. To stay with the example of coughing, a cough event usually generates an acoustic signal, an acceleration signal, an electrocardiogram signal, and other metrologically detectable signals that can be used for detection, identification and/or characterization of the event causing and/or accompanying the signals. The acoustic signal, the acceleration signal and the electromyographic signal are examples of signals of different modalities.

The basis for the procedures described herein are one or more signals of one or more modalities which relate to an event. Usually, the one or more signals are caused by the event or are accompanying symptoms of the event or cause the event.

Usually but not necessarily, the one or more signals relate to the same event. An “event” is a recognizable occurrence at a specific time or during a specific time period. An event can e.g. be a (specific) body action. An objective of the present invention can be to determine, for an event, whether the event is a body action and/or a specific body action and/or to determine which body action (specific or non-specific) the event is or relates to.

In a preferred embodiment of the present disclosure, the aim is to detect cough events and/or to identify cough events and/or to characterize cough events.

In a preferred embodiment of the present disclosure, at least one signal of the one or more signals is a sound signal (also referred to as audio signal), an electromyographic signal, an electrocardiogram signal, an accelerometer signal, a chest impedance signal, a temperature signal, and/or a plethysmographic signal.

“Sounds” are pressure variations in the air (or any other medium) that can be converted into an electrical signal with the help of a microphone and recorded mechanically or digitally. Other terms that are used for the term “sound” are “acoustic wave(s)” and “sound wave(s)”, which indicate that the pressure variations propagate through a transmission medium such as air.

An “audio recording” is a representation of one or more sounds that can be used to analyze and/or reproduce the one or more sounds. In other words: sound can be captured in an audio recording so that it can be analyzed and/or played back as often as required at a later point in time and/or at another location.

The term “audio” indicates that the sound is usually a pressure variation that is within a range that is audible to (can be heard by) the human ear. The human hearing range is commonly given as 20 to 20,000 Hz, although there is considerable variation between individuals. However, the term “audio” should not be understood to mean that the methods described herein are limited to sound waves in the range of 20 to 20,000 Hz. In principle, the methods presented here can also be applied to sound waves that are outside the range perceived by humans.

For capturing sound(s) as an audio signal, one or more microphones can be used. A microphone is a transducer that converts sound into an electrical signal. Several types of microphone can be used, which employ different methods to convert the air pressure variations of a sound wave to an electrical signal. The most common are dynamic microphones, which use a coil of wire suspended in a magnetic field, condenser microphones, which use a vibrating diaphragm as a capacitor plate, and contact microphones, which use a crystal of piezoelectric material. Microphones typically need to be connected to an amplifier before the signal can be recorded or reproduced.

The one or more microphones used to capture sound and convert it into an electric signal can be worn on the (human) body as so called “wearables”; they can be part of a device that a person carries with him/her, such as a mobile phone or a wrist watch; and/or they can be installed stationary in one or more rooms in which a person is occasionally or frequently present.

The electrical signal generated by the microphone(s) can be converted by an analog-to-digital converter into a digital signal. The digital signal can then be stored as an audio recording on a data storage.

One or more microphones, optionally one or more (pre-)amplifiers, one or more analog-to-digital converters and one or more data storages can be part of one device, or they can be part of separate devices which are connected to one another in order to generate audio recordings as described herein.

The audio recording can be saved in various audio file formats, e.g. in uncompressed waveform formats such as the waveform audio file format (WAV) and/or the audio interchange file form (AIFF), and/or with lossless compression such as FLAC (free lossless audio codes), and or with lossy compression such as MP3.

Details about generating audio recordings can be found in various textbooks (see e.g. F. Rumsey: Sound Recording—Application and Theory, 8^{th} edition, Focal Press, 2021, ISBN 9780367553029).

Each audio recording has a defined length, i.e. the time that elapses when the audio recording is played, and the sound(s) contained in the audio recording is/are reproduced.

Preferably, audio recordings with a (pre-)defined length are used. The length is preferably greater than the length of the sound to be detected and/or characterized. The length is preferably in the range of 1 second to 20 seconds, more preferably in the range of 2 seconds to 10 seconds.

If the length of an audio recording exceeds the defined length, it can be divided into sections having the defined length.

The division of an audio recording into sections is preferably carried out in such a way that in each subdivided section there is an area which is also contained in an adjacent section. In other words: if the sections are re-merged, in order to reconstruct the original audio file, there are overlapping areas. The degree of overlap of a section with an adjacent section can be e.g. 10%, 20%, 25%, 30%, 40%, 50% or any other percentage.

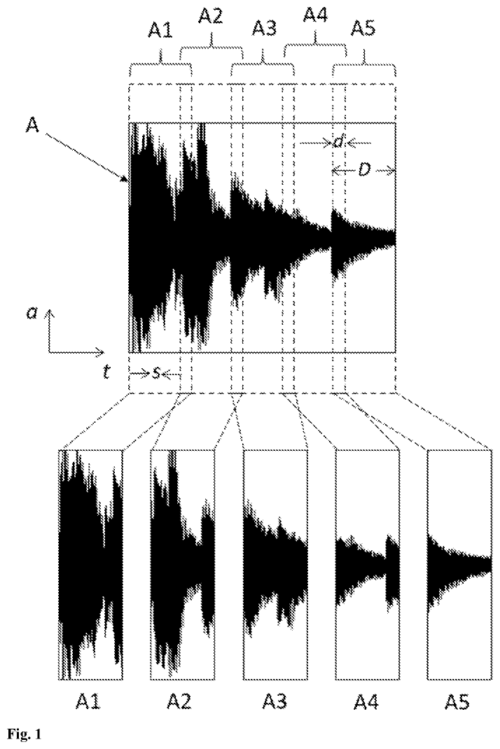

The process of dividing an audio recording into partially overlapping sections is schematically shown in FIG. 1. The audio recording A is displayed in the form of a time-domain representation (amplitude a as a function of time t). It is divided into 5 sections A1, A2, A3, A5, and A5. Section A1 contains an area which is also present in section A2, A2 contains areas which are also present in A1 and A3, A3 contains areas which are also present in A2 and A4, A4 contains areas which are also present in A3 and A5, and A5 contains an area which is also present in A4. In the example shown in FIG. 1, the degree of overlap of each section with each of the adjacent section is the same for all sections. The degree of overlap is given by the ratio d/D which is about 20% in case of the example shown in FIG. 1.

The division of an audio recording into partially overlapping sections can also be described with the help of a window that is stepwise moved along the time axis (see FIG. 1): A window is placed at the beginning of the audio recording. The window has a width D in the direction of the time axis t. The height of the window corresponds to the amplitude range (minimum to maximum) of the audio signal recorded in the audio recording. The area of the audio signal that lies within the window is the first section A1. Then the window will be moved a step along the time axis. The step size (window stride) is s=D−d. The area of the audio signal that lies now within the window is the second section A2. This process of stepwise moving the window is repeated until the window reaches the end of the audio signal.

By generating partially overlapping sections it can be prevented that cuts run through a sound of interest. By dividing a sound of interest, the sound is only incompletely present in the resulting sections and is therefore no longer clearly detectable and/or characterizable.

Instead of dividing an audio recording, the representations to be generated from the audio recording, as described in more detail hereinafter, can be generated from the original (undivided) audio recording in such a way that they have the defined length and/or partially overlap.

Preferably, each audio recording which is used for training of the machine learning model and/or for making predictions using the trained machine learning model, as described herein, is characterized by the same resolution, i.e. the same sampling rate (quantization of time) and the same bit depth (quantization of amplitude). The sampling rate usually lies in the range of 8 kHz to 384 kHz. The bit depth usually lies in the range of 8 bits to 128 bits. If an audio recording has a different resolution, it can be converted to the desired resolution by up-sampling or down-sampling.

What is described herein with respect to sound signals (audio signals) applies analogously to other signals that can be detected in the form of a physical quantity as a function of time. For example, an electromyographic signal is usually a measured voltage as a function of time (see e.g. A. Takano et al.: Non-Contact Measurements of Electrocardiogram and Cough-Associated Electromyogram from the Neck Using In-Pillow Common Cloth Electrodes: A Proof-of-Concept Study, 2021, Sensors 21(3):812, in particular FIG. 4). An electromyographic signal can be captured by a sensor (such as an electromyograph), recorded, digitized, and stored. The (digitized) signal can be divided into sections of defined length. It is possible to generate partially overlapping sections.

From each signal representing an event, at least two different representations are generated: a time-domain representation, and a spectrogram representation.

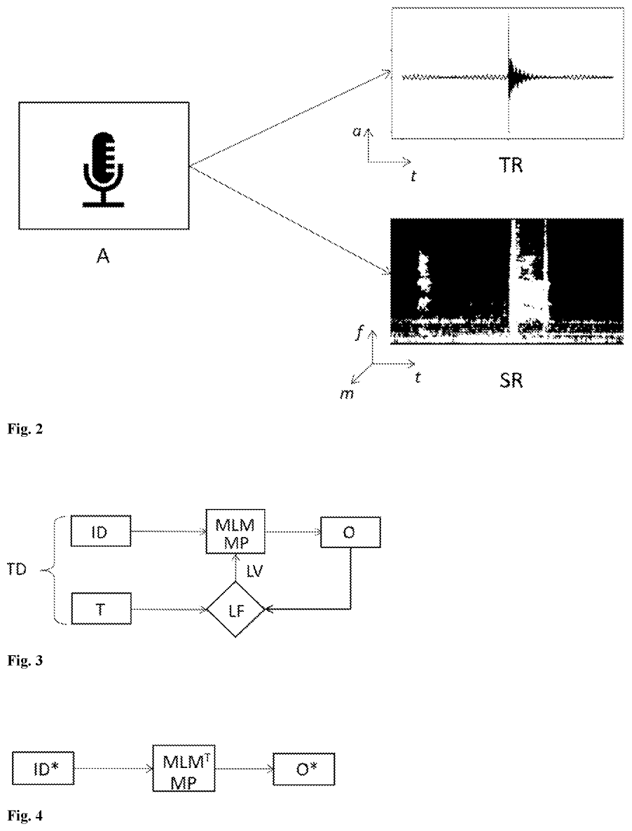

A “time-domain representation” represents a physical quantity as a function of time. In case of an audio signal, for example, the time-domain representation represents the loudness (amplitude) of captured sound(s) as a function of time. Examples of time-domain representations of captured (cough) sounds are given e.g. in FIGS. 1, 2 and 8 of US2020015709A1, and Figs. 1 and 2 of DOI:10.1109/ACCESS.2020.3018028.

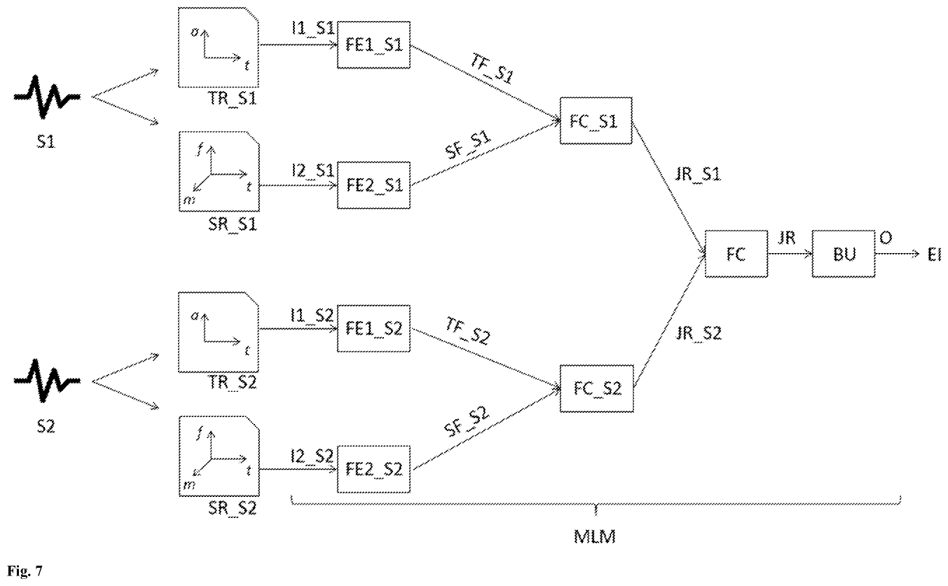

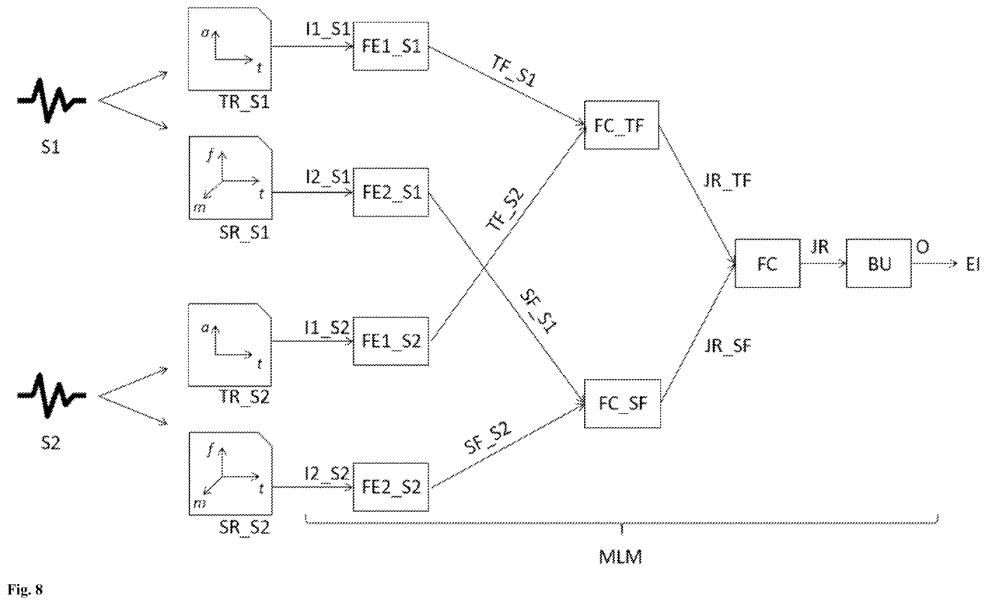

A “spectrogram representation” represents the intensities of frequency components as a function of time. In case of an audio signal, for example, the spectrogram representation represents the intensities of frequency components of a sound as a function of time. A spectrogram can be plotted e.g. as a three-dimensional graphic in which one axis represents the time (t), the second axis represents frequencies (f), and the third axis represent magnitude (m) of the observed frequencies at a particular time. Very often a spectrogram is displayed as a two-dimensional image in which one image axis represents the time, the other image axis represents frequencies and the colors or grey values represent magnitude (amplitude) of the observed frequencies at a particular time. Examples of spectrogram representations of captured (cough) sounds are given e.g. in Figs. 1 and 2 of DOI:10.1186/1745-9974-2-1 and FIGS. 7 and 8 of U.S. Pat. No. 6,436,057. Sometimes, a spectrogram is also referred to as time-frequency representation.

So, in case the one or more signals comprise a sound signal, at least two different representations are generated from the audio recording: a time-domain representation, and a spectrogram representation. Each representation is a representation of the respective audio signal contained in the audio recording.

The time-domain representation of the sound signal is usually characterized by a sampling rate in the range of 8 kHz to 384 kHz, and a bit depth in the range of 8 bits to 128 bits.

Methods for generating time-domain representations and spectrogram representations from audio recordings and other signals are known and are described in the publications cited herein and/or are disclosed in various textbooks (see e.g.: P. Hill: Audio and Speech processing with MATLAB, CRC Press, 2018, ISBN: 9780429813962).

FIG. 2 shows schematically by way of example, the generation of a time-domain representation TR and a spectrogram representation SR from an audio recording A. In the example shown in FIG. 2, both, the time-domain representation TR, and the spectrogram representation SR are generated from the audio recording A. However, it is also possible to first generate a time-domain representation from the audio recording and then use the time-domain representation in order to generate the spectrogram representation.

A spectrogram representation can e.g. be generated from a time-domain representation by short-time Fourier transformation (STFT). In practice, the procedure for computing a short-time Fourier transform is to divide a longer time signal into shorter segments (also referred to as frames or windows) preferably of equal width and then compute the Fourier transform separately on each shorter segment. This reveals the Fourier spectrum on each shorter segment. A plot of the changing spectra as a function of time reveals the spectrogram representation.

Preferably, when dividing the time signal into shorter segments, partially overlapping segments are generated as described above for the division of the audio recording into partially overlapping sections. Preferably, the window width is in the range of 10 ms to 50 ms. Preferably, the window stride is in the range of 1 ms to 8 ms.

The audio recording and/or the time-domain representation and/or the spectrogram representation can be analyzed to determine whether the recorded signal is of minimal intensity, e.g. in order to sort out audio recordings which do not contain any sound and/or which contain sound which is too quite to be analyzed and/or which contain sound that is so low that it is very likely not caused by cough (or any other body action). This analysis is preferably carried out on the basis of the audio recording sections. It is possible to compare the amplitude values with a pre-defined threshold. Audio recordings with amplitude values below the threshold can be discarded. The same applies to any other signal. Any signal or any time-domain representation and/or spectrogram representation of a signal can be pre-processed in order to sort out signals or sections within signals which do not contain information for detection and/or identification and/or characterization of a (specific) body action.

The time-domain representation and the spectrogram representation of each signal (and optionally additional input data as described herein) are used as input data for a machine learning model.

The time-domain representation and the spectrogram representation of each signal (and optionally any additional input data) can be used to train the machine learning model and to use the trained machine learning model to detect and/or identify and/or characterize a (specific) body action.

Such a “machine learning model”, as used herein, may be understood as a computer implemented data processing architecture. The machine learning model can receive input data and provide output data based on that input data and the machine learning model, in particular parameters of the machine learning model. The machine learning model can learn a relation between input data and output data through training. In training, parameters of the machine learning model may be adjusted in order to provide a desired output for a given input.

The output of the machine learning model can be a classification result, a regression result, a segmentation result and/or another result as described herein.

The process of training a machine learning model involves providing a machine learning algorithm (that is the learning algorithm) with training data to learn from. The term “trained machine learning model” refers to the model artifact that is created by the training process. The training data must contain the correct answer, which is referred to as the target. The learning algorithm finds patterns in the training data that map input data to the target, and it outputs a trained machine learning model that captures these patterns.

The training data comprise a multitude of reference signals, for each reference signal it is known to which body action it relates (which body action it represents).

The term “multitude” as it is used herein means an integer greater than 1, usually greater than 10, preferably greater than 100.

In case of detecting and/or identifying and/or characterizing (specific) cough events, for example, the training data comprise a multitude of reference audio recordings, and, for each reference audio recording, an information whether the reference audio recording is an audio recording of a cough or not (in case of the output being e.g. a binary classification result), and/or, depending on the desired output, an information about the type of cough (in case of the output being e.g. a classification with regard to the type of cough).

In the training process, training data are inputted into the machine learning model and the machine learning model generates an output. The output is compared with the (known) target. Parameters of the machine learning model are modified in order to reduce the deviations between the output and the (known) target to a (defined) minimum.

In general, a loss function can be used for training to evaluate the machine learning model. For example, a loss function can include a metric of comparison of the output and the target. The loss function may be chosen in such a way that it rewards a wanted relation between output and target and/or penalizes an unwanted relation between an output and a target. Such a relation can be e.g. a similarity, or a dissimilarity, or another relation.

A loss function can be used to calculate a loss value for a given pair of output and target. The aim of the training process can be to modify (adjust) parameters of the machine learning model in order to reduce the loss value to a (defined) minimum.

A loss function may for example quantify the deviation between the output of the machine learning model for a given input and the target. If, for example, the output and the target are numbers, the loss function can be the difference between these numbers, or the absolute value of the difference. In this case, a high absolute value of the loss function can mean that a parameter of the model needs to undergo a strong change.

In the case of a scalar output, a loss function may be a difference metric such as an absolute value of a difference, a squared difference.

In the case of vector-valued outputs, for example, difference metrics between vectors such as the root mean square error, a cosine distance, a norm of the difference vector such as a Euclidean distance, a Chebyshev distance, an Lp-norm of a difference vector, a weighted norm or any other type of difference metric of two vectors can be chosen. These two vectors may for example be the desired output (target) and the actual output.

In the case of higher dimensional outputs, such as two-dimensional, three-dimensional or higher-dimensional outputs, for example an element-wise difference metric may be used. Alternatively or additionally, the output data may be transformed, for example to a one-dimensional vector, before computing a loss function.

FIG. 3 shows schematically by way of example the process of training a machine learning model. The machine learning model MLM is trained on the basis of training data TD. The training data comprise a multitude of data sets, each data set comprising input data ID and target data T. In the example shown in FIG. 3, only one data set comprising input data ID and a target T is shown. The input data ID is inputted into the machine learning model MLM. The machine learning model is configured to generate, at least partially on the basis of the input data ID and model parameters MP, an output O. The output O is compared with the target T. This is done by using a loss function LF, the loss function quantifying the deviations between the output O and the target T. For each pair of an output O and the respective target T, a loss value is computed using the loss function LF. During training the model parameters are modified in a way that reduces the loss values to a defined minimum. The aim of the training is to let the machine learning model generate for each input data an output which comes as close to the corresponding target as possible. Once the defined minimum is reached, the (now fully trained) machine learning model can be used to predict an output for new input data (input data which have not been used during training and for which the target is usually not (yet) known).

FIG. 4 shows schematically by way of example how a trained machine learning model can be used for making predictions. The trained machine learning model (MLM^{T}) can be the machine learning model described with reference to FIG. 3. New input data ID* are inputted into the trained machine learning model (MLM^{T}). The trained machine learning model (MLM^{T}) is configured and trained to generate, at least partially on the basis of the new input data ID* and the (trained) model parameters MD, an output O*.

The machine learning model according to the present disclosure comprises an output, and, for each signal processed by the machine learning model, at least two inputs, a first input and a second input.

It is possible that the machine learning model comprises one or more additional inputs and/or one or more additional outputs.

The first input is configured to receive the time-domain representation of a respective signal, and the second input is configured to receive the spectrogram representation of the respective signal.

The output is configured to output a classification result, a regression result, a segmentation result and/or another result as described herein.

The machine learning model according to the present disclosure further comprises, for each signal, a first feature extraction unit and a second feature extraction unit. The first feature extraction unit is configured to generate time-domain features at least partially on the basis of the time-domain representation of a respective signal. The second feature extraction unit is configured to generate spectrogram features at least partially on the basis of the spectrogram representation of the respective signal.

Feature extraction from the time-domain representation and/or the spectrogram representation is usually accompanied by a reduction in dimensions. In other words: from a time-domain representation time-domain features are generated, from a spectrogram representation spectrogram features are generated, and the time-domain features as a whole have less dimensions than the time-domain representation, and the spectrogram features as a whole have less dimensions than the spectrogram representation. In other words: during training, the machine learning model is trained to extract those features from each time-domain representation and each spectrogram representation that have a significant influence on the output/target (that are responsible for the output/target).

Both, the time-domain features and the spectrogram features, are usually in the form of one or more feature vectors.

In machine learning, a feature vector is a p-dimensional vector of numerical features that represent an object (in this case an audio signal), wherein p is an integer greater than 0. The term “feature vector” shall also include single values, matrices, tensors, and the like. The time-domain features and the respective time-domain feature vector are (condensed) numerical representations of the time-domain representation of a respective signal. The spectrogram features and the respective spectrogram feature vector are (condensed) representations of the spectrogram representation of the respective signal.

Examples of feature extraction and feature vector generation methods can be found in various textbooks and scientific publications (see e.g. G. A. Tsihrintzis, L. C. Jain: Machine Learning Paradigms: Advances in Deep Learning-based Technological Applications, in: Learning and Analytics in Intelligent Systems Vol. 18, Springer Nature, 2020, ISBN: 9783030497248; K. Grzegorczyk: Vector representations of text data in deep learning, Doctoral Dissertation, 2018, arXiv:1901.01695v1 [cs.CL]; M. Ilse et al.: Attention-based Deep Multiple Instance Learning, arXiv:1802.04712v4 [cs.LG]).

The machine learning model according to the present disclosure further comprises a feature combination unit. The feature combination unit is configured to generate, on the basis of all time-domain features and spectrogram features (and optionally on the basis of additional features) a joint representation. In the joint representation, the time-domain features and the spectrogram features (and optionally the additional features) are combined. The term “combination” as it is used herein means the combination of two or more pieces of data into a single piece of data. For example, two vectors may be concatenated to a single vector.

When two or more originally disjoint pieces of data are combined to one piece of data, they form a joint representation. Such a joint representation may comprise (e.g., contain) essentially e.g. all or most of the information comprised (e.g., contained) in the individual pieces of data that the joint representation was derived of. The joint representation may also comprise (e.g., contain) a part of the information included in both or one of the individual pieces of data.

Combination(s) of (e.g. at least two) representations to generate a joint representation may in case of vectors and/or matrices and/or tensors for example be achieved by a multiplication, an addition, for example an element-wise addition, a cross-product, a concatenation, a stacking on top of each other, and many other procedures.

The choice of (mathematical) operation for the combination of two or more features to generate a joint representation may depend at least partially on their representations, be it their respective initial representations as input data or their respective transformed intermediate representation (generated by the feature extraction units). As an example, in the case that the different pieces of data to be combined, are represented as matrices, tensors or other arrays with two or more dimensions they may be combined (directly, if the dimensions allow, or after rearranging the matrix in order to achieve fitting dimensions), multiplied either in total or element-wise, added, interlaced, entries from a matrix representations may be partially or entirely taken over into the joint representation, or a combination thereof, to name but a few non-limiting examples. In case of two or more vector representations, similar combination operations may be used such as concatenation, multiplication, addition, cross products, stacking, or a combination thereof, to name but a few non-limiting examples.

It may often be desirable that the combination operation between two (or more) features fulfills certain criteria. The combination may for example preserve the property of differentiability. This is to mean that the input-output relation between any single value of a feature vector before combining (e.g., an entry of a scalar, vector, matrix, tensor or the like) and any single value of the joint representation allows the computation of a derivative. For example, a derivative of the entry in the joint representation may be computed with respect to the entries in the “separate” representations before combining to a joint representation. This property may for example be valuable in training where a derivative of the loss function with respect to one or multiple or even all parameters of the model is used. Differentiability of the combination functionality may be beneficial e.g. when the training of the machine learning model involves backpropagation. As an example, if two matrices {x1ij} and {x2ij} are combined to a matrix {yij} forming the joint representation, a respective entry of the joint representation matrix yij can either be an entry x1ij of the first matrix or an entry x2ij of the second matrix. The combination function per joint matrix entry is thus yij=aij*x1ij+(1−aij)*x2ij with a∈{0, 1}. Thus, such a combination preserves differentiability as the function yij=x1ij is differentiable with respect to x1ij, for example. A matrix multiplication that per element involves an element-wise multiplication of a line of a first matrix and a column of a second matrix followed by a sum also preserves differentiability. Many different other ways of combining two vectors or matrices exist that may preserve the quality of differentiability.

The machine learning model according to the present disclosure further comprises a body action detection, identification and/or characterization unit. The body action detection, identification and/or characterization unit is configured to determine, on the basis of the joint representation, whether the one or more signals related to an event are caused by or related to a (specific) body action and/or what type of body action is represented by the one or more signals and/or which areas in the one or more a signals represent (a specific type of) body action. The result(s) of the determination performed by the body action detection, identification and/or characterization unit (also referred to as event information) is outputted via the output of the machine learning model.

The result (event information) provided by the machine learning model can be displayed on a monitor, printed on a printer, transmitted to another computer system, and/or stored on a data storage.

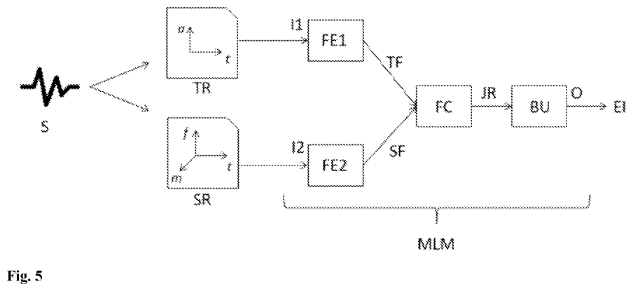

FIG. 5 shows schematically by way of example a preferred architecture of the (trained) machine learning model. The example depicted in FIG. 5 represents a (trained) machine learning model for processing a single signal S. From the signal S, a time-domain representation TR and a spectrogram representation SR are generated. The (trained) machine learning model MLM comprises a first input I1, a second input I2, a first feature extraction unit FE1, a second feature extraction unit FE2, a feature combination unit FC, a body action detection, identification and/or characterization unit BU, and an output O.

The first input I1 is provided by the first feature extraction unit FE1. The first input I1 is configured to receive the time-domain representation TR of the signal S. The first feature extraction unit FE1 is configured to determine time-domain features TF on the basis of the time-domain representation TR.

The second input I2 is provided by the second feature extraction unit FE2. The second input I2 is configured to receive a spectrogram representation SR of the signal S. The second feature extraction unit FE2 is configured to determine spectrogram features SF on the basis of the spectrogram representation SR.

The feature combination unit FC is configured to generate, at least partially on the basis of the time-domain features TF and the spectrogram features SF, a joint representation JR.

The body action detection, identification and/or characterization unit BU is configured to generate, at least partially on the basis of the joint representation JR, an event information EI. The event information EI can be outputted via the output O.

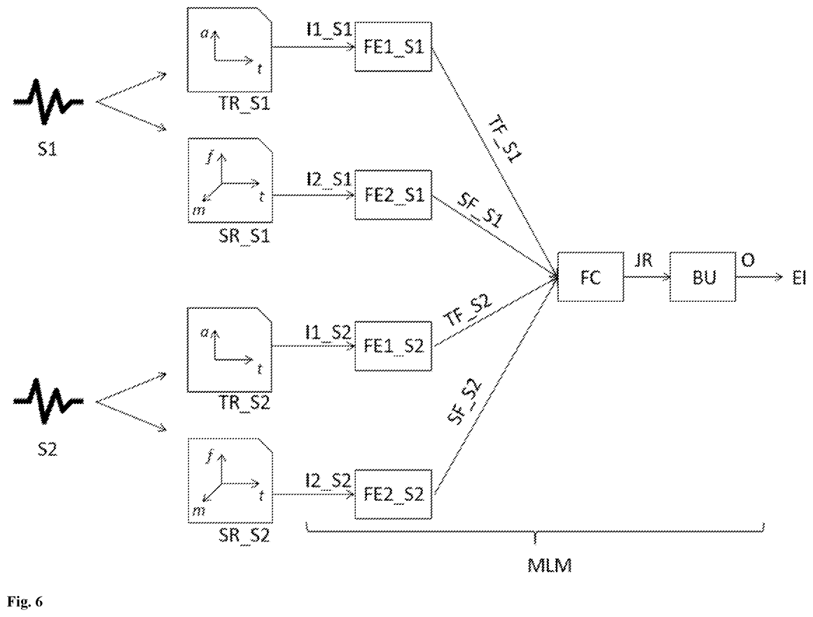

FIG. 6 shows schematically by way of example another preferred architecture of the (trained) machine learning model. The example depicted in FIG. 6 represents a (trained) machine learning model for processing two signals S1 and S2. From each signal, a time-domain representation and a spectrogram representation are generated: from the first signal S1, a time-domain representation TR_S1 and a spectrogram representation SR_S1 are generated; from the second signal S2, a time-domain representation TR_S2 and a spectrogram representation SR_S2 are generated.

The (trained) machine learning model MLM comprises for each signal (S1, S2), a first input (I1_S1, I1_S2), a second input (I2_S1, I2_S2), a first feature extraction unit (FE1_S1, FE1_S2), and a second feature extraction unit (FE2_S1, FE2_S2).

The (trained) machine learning model MLM further comprises a feature combination unit FC, a body action detection, identification and/or characterization unit BU, and an output O.

The first input I1_S1 for the first signal S1 is provided by the first feature extraction unit FE1_S1. The first input I1_S1 is configured to receive the time-domain representation TR_S1 of the first signal S1. The first feature extraction unit FE1_S1 is configured to determine time-domain features TF_S1 on the basis of the time-domain representation TR_S1.

The first input I1_S2 for the second signal S2 is provided by the first feature extraction unit FE1_S2. The first input I1_S2 is configured to receive the time-domain representation TR_S2 of the second signal S2. The first feature extraction unit FE1_S2 is configured to determine time-domain features TF_S2 on the basis of the time-domain representation TR_S2.

The second input I2_S1 for the first signal S1 is provided by the second feature extraction unit FE2_S1. The second input I2_S1 is configured to receive a spectrogram representation SR_S1 of the first signal S1. The second feature extraction unit FE2_S1 is configured to determine spectrogram features SF_S1 on the basis of the spectrogram representation SR_S1.

The second input I2_S2 for the second signal S2 is provided by the second feature extraction unit FE2_S2. The second input I2_S2 is configured to receive a spectrogram representation SR_S2 of the second signal S2. The second feature extraction unit FE2_S2 is configured to determine spectrogram features SF_S2 on the basis of the spectrogram representation SR_S2.

The feature combination unit FC is configured to generate, at least partially on the basis of the time-domain features TF_S1, TF_S2 and the spectrogram features SF_S1, SF_S2, a joint representation JR.

The body action detection, identification and/or characterization unit BU is configured to generate, at least partially on the basis of the joint representation JR, an event information EI. The event information EI can be outputted via the output O.

FIG. 7 shows schematically by way of example another preferred architecture of the (trained) machine learning model. The example depicted in FIG. 7 represents a (trained) machine learning model for processing two signals S1 and S2. From each signal (S1, S2), a time-domain representation (TR_S1, TR_S2) and a spectrogram representation (SR_S1, SR_S2) are generated.

The (trained) machine learning model MLM comprises for each signal (S1, S2), a first input (1I_S1, I1_S2), a second input (I2_S1, I2_S2), a first feature extraction unit (FE1_S1, FE1_S2), and a second feature extraction unit (FE2_S1, FE2_S2).

The (trained) machine learning model MLM further comprises a first feature combination unit FC_S1 for features of the first signal S1, a second feature combination unit FC_S2 for features of the second signal S2, a third feature combination unit FC, a body action detection, identification and/or characterization unit BU, and an output O.

The first input I1_S1 is configured to receive the time-domain representation TR_S1 of the first signal S1. The first feature extraction unit FE1_S1 is configured to determine time-domain features TF_S1 on the basis of the time-domain representation TR_S1.

The first input I1_S2 is configured to receive the time-domain representation TR_S2 of the second signal S2. The first feature extraction unit FE1_S2 is configured to determine time-domain features TF_S2 on the basis of the time-domain representation TR_S2.

The second input I2_S1 is configured to receive a spectrogram representation SR_S1 of the first signal S1. The second feature extraction unit FE2_S1 is configured to determine spectrogram features SF_S1 on the basis of the spectrogram representation SR_S1.

The second input I2_S2 is configured to receive a spectrogram representation SR_S2 of the second signal S2. The second feature extraction unit FE2_S2 is configured to determine spectrogram features SF_S2 on the basis of the spectrogram representation SR_S2.

The first feature combination unit FC_S1 is configured to generate, at least partially on the basis of the time-domain features TF_S1 and the spectrogram features SF_S1, a joint representation JR_S1.

The second feature combination unit FC_S2 is configured to generate, at least partially on the basis of the time-domain features TF_S2 and the spectrogram features SF_S2, a joint representation JR_S2.

The third feature combination unit FC is configured to generate, at least partially on the basis of the joint representation JR_S1 and the joint representation JR_S2, a joint representation JR.

The body action detection, identification and/or characterization unit BU is configured to generate, at least partially on the basis of the joint representation JR, an event information EI. The event information EI can be outputted via the output O.

FIG. 8 shows schematically by way of example another preferred architecture of the (trained) machine learning model. The example depicted in FIG. 8 represents a (trained) machine learning model for processing two signals S1 and S2. From each signal (S1, S2), a time-domain representation (TR_S1, TR_S2) and a spectrogram representation (SR_S1, SR_S2) are generated.

The (trained) machine learning model MLM comprises for each signal (S1, S2), a first input (1I_S1, I1_S2), a second input (I2_S1, I2_S2), a first feature extraction unit (FE1_S1, FE_S2), and a second feature extraction unit (FE2_S1, FE2_S2).

The (trained) machine learning model MLM further comprises a first feature combination unit FC_TF for the time-domain features TF_S1 of the first signal S1 and the time-domain features TF_S2 of the second signal S2, a second feature combination unit FC_SF for spectrogram features SF_S1 of the first signal S1 and the spectrogram features SF_S2 of the second signal S2, a third feature combination unit FC, a body action detection, identification and/or characterization unit BU, and an output O.

The first input I1_S1 is configured to receive the time-domain representation TR_S1 of the first signal S1. The first feature extraction unit FE1_S1 is configured to determine time-domain features TF_S1 on the basis of the time-domain representation TR_S1.

The first input I1_S2 is configured to receive the time-domain representation TR_S2 of the second signal S2. The first feature extraction unit FE1_S2 is configured to determine time-domain features TF_S2 on the basis of the time-domain representation TR_S2.

The second input I2_S1 is configured to receive a spectrogram representation SR_S1 of the first signal S1. The second feature extraction unit FE2_S1 is configured to determine spectrogram features SF_S1 on the basis of the spectrogram representation SR_S1.

The second input I2_S2 is configured to receive a spectrogram representation SR_S2 of the second signal S2. The second feature extraction unit FE2_S2 is configured to determine spectrogram features SF_S2 on the basis of the spectrogram representation SR_S2.

The first feature combination unit FC_TF is configured to generate, at least partially on the basis of the time-domain features TF_S1 and TF_S2, a joint representation JR_TF.

The second feature combination unit FC_SF is configured to generate, at least partially on the basis of the spectrogram features SF_S1 and SF_S2, a joint representation JR_SF.

The third feature combination unit FC is configured to generate, at least partially on the basis of the joint representation JR_TF and the joint representation JR_SF, a joint representation JR.

The body action detection, identification and/or characterization unit BU is configured to generate, at least partially on the basis of the joint representation JR, an event information EI. The event information EI can be outputted via the output O.

As described above, FIGS. 7 and 8 show preferred architectures of a (trained) machine learning model for processing two signals S1 and S2. In case of further signals (e.g. a third signal S3), from each further signal a time-domain representation and a spectrogram representation can be generated. Each further time-domain representation is inputted into an additional input of the machine learning model, and each spectrogram representation is inputted into an additional input. In case of n signals, each signal preferably being of a different modality, there are 2-n inputs, n being an integer equal to or greater than 1. So, for example, in case of 3 signals, there are 6 inputs; in case of 4 signals, there are 8 inputs. There can be additional inputs for additional input data as described herein. From each time-domain representation and each spectrogram representation inputted into the machine learning model, feature vectors are generated by feature extraction units. The feature vectors are combined to joint representations. The combination can be done stepwise, as depicted in FIGS. 7 and 8, or concertedly, as depicted in FIG. 6. In case of more than two signals, combinations of the combination procedures as shown in FIGS. 6 to 8 are possible.

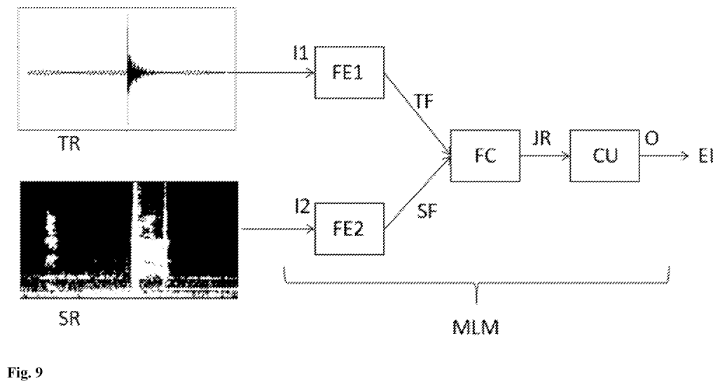

FIG. 9 shows schematically by way of example a preferred architecture of the (trained) machine learning model for detection, identification and/or characterization of cough events. The machine learning model MLM comprises a first input I1, a second input I2, a first feature extraction unit FE1, a second feature extraction unit FE2, a feature combination unit FC, a cough detection, identification and/or characterization unit CU, and an output O.

The first input I1 is provided by the first feature extraction unit FE1. The first input I1 is configured to receive a time-domain representation TR of an audio recording. The first feature extraction unit FE1 is configured to determine time-domain features TF on the basis of the time-domain representation TR.

The second input I2 is provided by the second feature extraction unit FE2. The second input I2 is configured to receive a spectrogram representation SR of the audio recording. The second feature extraction unit FE2 is configured to determine spectrogram features SF on the basis of the spectrogram representation SR.

The feature combination unit FC is configured to generate, at least partially on the basis of the time-domain features TF and the spectrogram features SF, a joint representation JR.

The cough detection, identification and/or characterization unit CU is configured to generate, at least partially on the basis of the joint representation JR, an event information EI. The event information EI can be outputted via the output O.

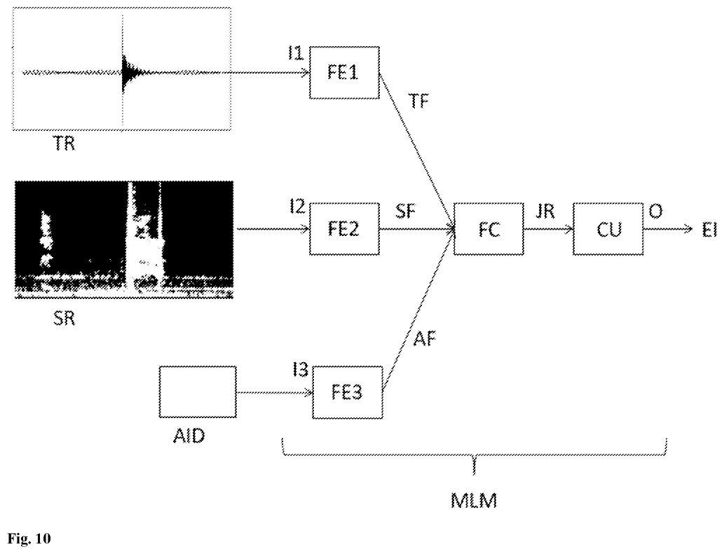

FIG. 10 shows schematically by way of example another preferred architecture of the (trained) machine learning model for the detection, identification and/or characterization of cough events. While the machine learning model depicted in FIG. 9 has two inputs and two feature extraction units, the machine learning model MLM depicted in FIG. 10 has three inputs and three feature extraction units.

The first input I1 is provided by the first feature extraction unit FE1 and is configured to receive a time-domain representation TR of an audio recording. The first feature extraction unit FE1 is configured to determine time-domain features TF on the basis of the time-domain representation TR.

The second input I2 is provided by the second feature extraction unit FE2 and is configured to receive a spectrogram representation SR of the audio recording. The second feature extraction unit FE2 is configured to determine spectrogram features SF on the basis of the spectrogram representation SR.

The third input I3 is provided by the third feature extraction unit FE3 and is configured to receive additional input data AID. The third feature extraction unit FE3 is configured to determine additional features AF on the basis of the additional input data AID.

The feature combination unit FC is configured to generate, at least partially on the basis of the time-domain features TF, the spectrogram features SF, and the additional features AF, a joint representation JR.

The cough detection, identification and/or characterization unit CU is configured to generate, at least partially on the basis of the joint representation JR, an event information EI. The event information EI can be outputted via the output O.

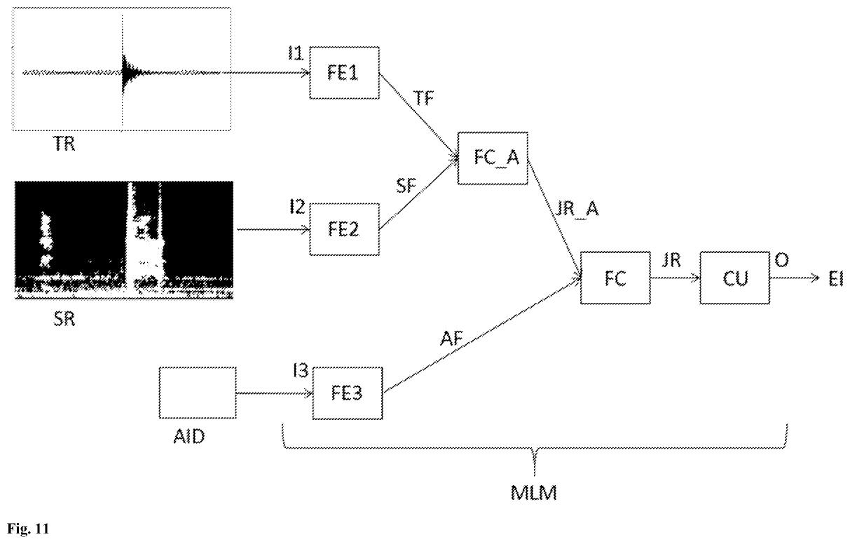

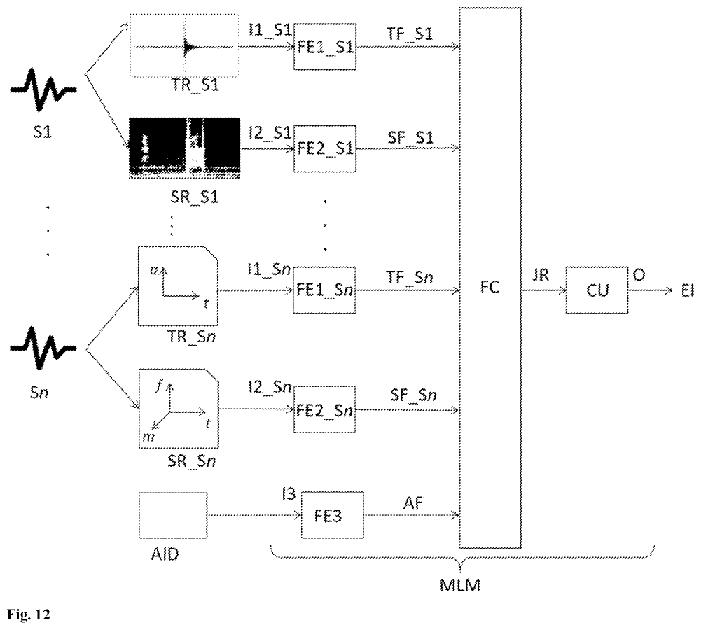

FIG. 11 shows schematically by way of example another preferred architecture of the (trained) machine learning model for the detection, identification and/or characterization of cough events. The (trained) machine learning model (MLM) comprises a first input I1 provided by a first feature extraction unit FE1, and a second input I2 provided by a second feature extraction unit FE2.

The first input I1 is configured to receive a time-domain representation TR of an audio recording. The first feature extraction unit FE1 is configured to determine time-domain features TF on the basis of the time-domain representation TR.

The second input I2 is configured to receive a spectrogram representation SR of the audio recording. The second feature extraction unit FE2 is configured to determine spectrogram features SF on the basis of the spectrogram representation SR.

The (trained) machine learning model (MLM) further comprises a third input I3 provided by the third feature extraction unit FE3. The third input I3 is configured to receive additional input data AID. The third feature extraction unit FE3 is configured to determine additional features AF on the basis of the additional input data AID.

The (trained) machine learning model (MLM) further comprises a first feature combination unit FC_A. The first feature combination unit FC_A is configured to generate, at least partially on the basis of the time-domain features TF, and the spectrogram features SF, a joint representation JR_A.

The (trained) machine learning model (MLM) further comprises a feature combination unit FC which is configured to generate, at least partially on the basis of the joint representation JR_A, and the additional features AF, a joint representation JR.

The cough detection, identification and/or characterization unit CU is configured to generate, at least partially on the basis of the joint representation JR, an event information EI. The event information EI can be outputted via the output O.

FIG. 12 shows schematically by way of example another preferred architecture of the (trained) machine learning model for the detection, identification and/or characterization of cough events. The example depicted in FIG. 12 represents a (trained) machine learning model for processing a number of n signals S1 to Sn, and additional input data AID, wherein n is an integer greater than 1.

The first signal S1 is preferably a sound signal (audio signal). Each of the n further signals can be selected from the following list: an electromyographic signal, an electrocardiogram signal, an accelerometer signal, a chest impedance signal, a plethysmographic signal, a temperature signal, a heart rate signal, a blood pressure signal.

From each signal S1 to Sn a time-domain representation (TR_S1 to TR_Sn) and a spectrogram representation (SR_S1 to SR_Sn) is generated. The machine learning model (MLM) comprises, for each signal S1 to Sn, a first input (I1_S1 to I1_Sn) and a second input (I2_S1 to I2_Sn). The machine learning model (MLM) further comprises a third input I3 for the additional input data AID.

The machine learning model further comprises, for each signal S1 to Sn, a first feature extraction unit (FE1_S1 to FE1_Sn) and a second feature extraction unit (FE2_S1 to FE2_Sn). The machine learning model (MLM) further comprises a third feature extraction unit FE3.

Each first input (I1_S1 to I1_Sn) is configured to receive the time-domain representation of a respective signal. Each second input (I2_S1 to I2_Sn) is configured to receive the spectrogram representation of the respective signal.

The third input I3 is configured to receive the additional input data AID.

Each first feature extraction unit (FE1_S1 to FE1_Sn) is configured to generate, on the basis of the time-domain representation of the respective signal, time-domain features (TF_S1 . . . TF_Sn).

Each second feature extraction unit (FE2_S1 to FE2_Sn) is configured to generate, on the basis of the spectrogram representation of the respective signal, spectrogram features (SF_S1 . . . SF_Sn).

The third feature extraction unit FE3 is configured to generate, on the basis of the additional input data, additional features AF.

The machine learning model MLM further comprises a feature combination unit FC. The feature combination unit FC is configured to generate, at least partially on the basis of all features (TF_S1 . . . TF_Sn, SF_S1 . . . SF_Sn, AF), a joint representation JR. Combination can be performed stepwise, as e.g. shown in FIGS. 7, 8, and 11, or concertedly, as e.g. shown in FIGS. 6, and 10. Also combinations are possible; it is possible that two or more feature vectors are combined to a joint representation before the joint representation is combined with one or more further feature vectors or further joint representations to another joint representation.

The machine learning model MLM further comprises a cough detection, identification and/or characterization unit CU. The cough detection, identification and/or characterization unit CU is configured to generate, at least partially on the basis of the joint representation JR, an event information EI. The event information EI can be outputted via the output O.

It should be noted that the machine learning model of the present disclosure can also be a combination of the models shown in FIGS. 5 to 12.

The machine learning model can be set up as a classifier. A classifier sorts input data into one of several (pre-)defined groups (classes).

The machine learning model can e.g. be a binary classifier which classifies an event on the basis of one or more signals related to the event into one of two classes, a first class and a second class, the first class consisting of events which relate to a (specific) body action, the second class consisting of events which do not relate to a (specific) body action.

With regard to the detection of cough, this means: The machine learning model can be a binary classifier which classifies an audio recording into one of two classes, a first class and a second class, the first class consisting of audio recordings which contain one or more cough events, and the second class consisting of audio recordings which do not contain a cough event. Or: The machine learning model can be a binary classifier which classifies an audio recording into one of two classes, a first class and a second class, the first class consisting of audio recordings which contain a specific type of cough (e.g. pertussis), and the second class consisting of audio recordings which do not contain the specific type of cough (but may contain a different type of cough).

The machine learning model can be a classifier which classifies an event into one of a number m of classes, with m being an integer greater than 2, wherein at least m−1 classes of the number m of classes consist of body actions of a specific type. The m^{th} class may consist of events which do not contain any body action.

With regard to the detection of cough, this may mean: The machine learning model can be a classifier which classifies an audio recording into one of a number m of classes, with m being an integer greater than 2, wherein at least m−1 classes of the number m of classes consist of audio recordings of a specific type. The m^{th} class may consist of audio recordings which do not contain any cough sound.

The machine learning model can be a regression tool which determines, for each signal or group of signals related to an event and inputted into the machine learning model, a number, the number being indicative of the likelihood that the event contains a (specific) body action.

With regard to the detection of cough, this may mean: The machine learning model can be a regression tool which determines, for each audio recording inputted into the machine learning model, a number, the number being indicative of the likelihood that the audio recording contains a cough. Or: The machine learning model can be a regression tool which determines, for each audio recording inputted into the machine learning model, a number, the number being indicative of the number of coughs contained in the audio recording.

The machine learning model can be a segmentation tool which marks in each signal related to an event and inputted into the machine learning model one or more time intervals containing a (specific) body action (e.g. a cough event).

In a preferred embodiment of the present disclosure, the machine learning model outputs a vector, the vector containing a number of timesteps and for each time step a probability value, the probability value indicating the probability that the signal at the respective timestep is caused by or related to a (specific) body action (e.g. a cough event, see the Example below).

In a preferred embodiment, the number (counts) of coughs contained in an audio recording is determined for each audio recording inputted into the trained machine learning model. A sound recorded at a certain timestep is considered to be caused by a cough if the probability value of at least a pre-defined number of adjacent timesteps (e.g. 5 or 10 or 20 or any other number) is equal to or greater than a pre-defined threshold. The threshold can e.g. be 70%, 75%, 80%, 83%, 85%, 87%, 90%, 95% or any other percentage.

It is also possible to plot the probability values as a function of time (timesteps), optionally smoothen the function (e.g. by using a spline-function or another approximation function), and determine the peaks (local maxima). Peak maxima above a pre-defined threshold and/or peaks with a peak width (e.g. full width at half maximum) above a lower pre-defined threshold and/or below an upper pre-defined threshold can be considered as a sound created by a cough.

The number of coughs over a defined period (e.g. an hour, or 12 hours, or a day, or a couple of days) can be an important biomarker e.g. for disease recognition and/or characterization.

The machine learning model according to the present disclosure can be or comprise one or more artificial neural networks.

An artificial neural network (ANN) is a biologically inspired computational model. An ANN usually comprises at least three layers of processing elements: a first layer with input neurons (nodes), a kth layer with at least one output neuron (node), and k−2 inner (hidden) layers, where k is an integer greater than 2.

In such a network, the input neurons serve to receive the input data. If the input data constitute or comprise an image (e.g. a spectrogram representation), there is usually one input neuron for each pixel/voxel of the input image; there can be additional input neurons for additional input data such as data about measurement conditions, data about the subject which generated the body action and/or the like. The output neurons serve to output the output data (result(s)).

The processing elements of the layers are interconnected in a predetermined pattern with predetermined connection weights therebetween. Each network node usually represents a (simple) calculation of the weighted sum of inputs from prior nodes and a non-linear output function. The combined calculation of the network nodes relates the inputs to the outputs.

When trained, the connection weights between the processing elements in the ANN contain information regarding the relationship between the input data and the output data which can be used to predict new output data from a new input data.

Each network node represents a calculation of the weighted sum of inputs from prior nodes and a non-linear output function. The combined calculation of the network nodes relates the inputs to the output(s).

Separate networks can be developed for each property measurement or groups of properties can be included in a single network. Preferably, different dimensions and/or modalities of patient data (and/or optionally additional data) are combined in a joint representation.

Training estimates network weights that allow the network to calculate (an) output value(s) close to the measured output value(s). A supervised training method can be used in which the output data is used to direct the training of the network weights. The network weights can be initialized with small random values or with the weights of a prior partially trained network. The training data inputs are applied to the network and the output values are calculated for each training sample. The network output values are compared to the measured output values. A backpropagation algorithm can be applied to correct the weight values in directions that reduce the error between measured and calculated outputs. The process is iterated until no further reduction in error can be made or until a predefined prediction accuracy has been reached.

A cross-validation method can be employed to split the data into training and validation data sets. The training data set is used in the backpropagation training of the network weights. The validation data set is used to verify that the trained network generalizes to make good predictions. The best network weight set can be taken as the one that best predicts the outputs of the training data. Similarly, varying the number of network hidden nodes and determining the network that performs best with the data sets optimizes the number of hidden nodes.

In a preferred embodiment of the present invention, the machine learning model is or comprises one or more convolutional neural networks (CNN).

A CNN is a class of deep neural networks, most commonly applied to analyzing visual imagery (such as spectrogram representations). A CNN comprises an input layer with input neurons, an output layer with at least one output neuron, as well as multiple hidden layers between the input layer and the output layer.

The hidden layers of a CNN typically consist of convolutional layers, ReLU (Rectified Linear Units) layer i.e. activation function, pooling layers, fully connected layers and normalization layers.

The nodes in the CNN input layer are organized into a set of “filters” (feature detectors), and the output of each set of filters is propagated to nodes in successive layers of the network. The computations for a CNN include applying the convolution mathematical operation to each filter to produce the output of that filter. Convolution is a specialized kind of mathematical operation performed by two functions to produce a third function that is a modified version of one of the two original functions. In convolutional network terminology, the first function to the convolution can be referred to as the input, while the second function can be referred to as the convolution kernel. The output may be referred to as the feature map. For example, the input to a convolution layer can be a multidimensional array of data that defines the various color components or grey scale values of an input image. The convolution kernel can be a multidimensional array of parameters, where the parameters are adapted by the training process for the neural network.

The machine learning model may be or comprise one or more recurrent neural networks. Recurrent neural networks (RNNs) are a family of feedforward neural networks that include feedback connections between layers. RNNs enable modeling of sequential data by sharing parameter data across different parts of the neural network. The architecture for an RNN includes cycles. The cycles represent the influence of a present value of a variable on its own value at a future time, as at least a portion of the output data from the RNN is used as feedback for processing subsequent input in a sequence.

In a preferred embodiment of the present disclosure, one or more first feature extraction units and/or one or more second feature extraction units and/or any further feature extraction unit (if present) is or comprises a convolutional neural network. In a preferred embodiment of the present disclosure, the convolutional neural network of the first feature extraction unit(s) comprise(s) shortcut connections which allow residual mappings.

In a preferred embodiment of the present disclosure, the body action detection, identification and/or characterization unit is or comprises a recurrent neural network.

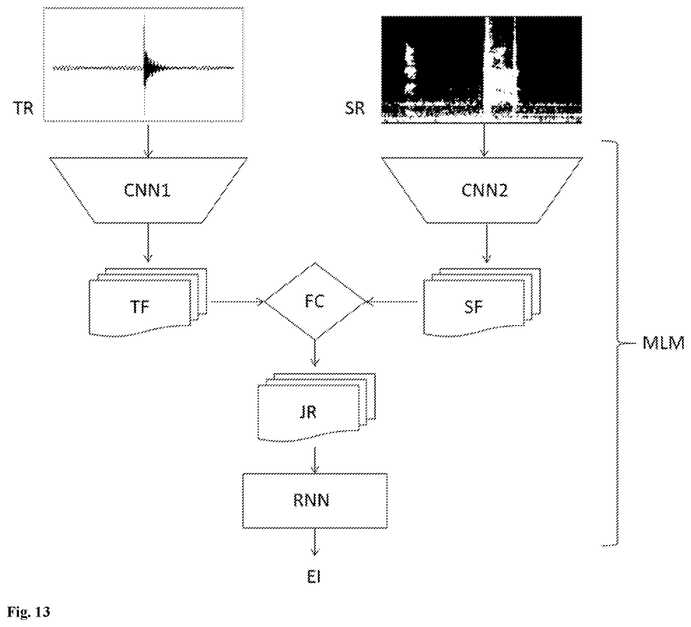

FIG. 13 shows schematically by way of example another preferred architecture of the (trained) machine learning model or a part thereof. The machine learning model MLM depicted in FIG. 13 is configured to detect, identify and/or characterize cough event.

The machine learning model MLM comprises a first convolutional neural network CNN1 which is configured to receive a time-domain representation TR of an audio recording, and to generate, at least partially on the basis of the time-domain representation TR, time-domain features TF.

The machine learning model MLM further comprises a second convolutional neural network CNN2 which is configured to receive a spectrogram representation SR of an audio recording, and to generate, at least partially on the basis of the spectrogram representation SR, spectrogram features SF.

The machine learning model MLM further comprises a feature combination unit FC which is configured to generate, at least partially on the basis of the time-domain features TF and the spectrogram features SF, a joint representation JR.

The machine learning model MLM further comprises a recurrent neural network RNN, the recurrent neural network RNN is configured to generate, at least partially on the basis of the joint representation JR, an event information EI.

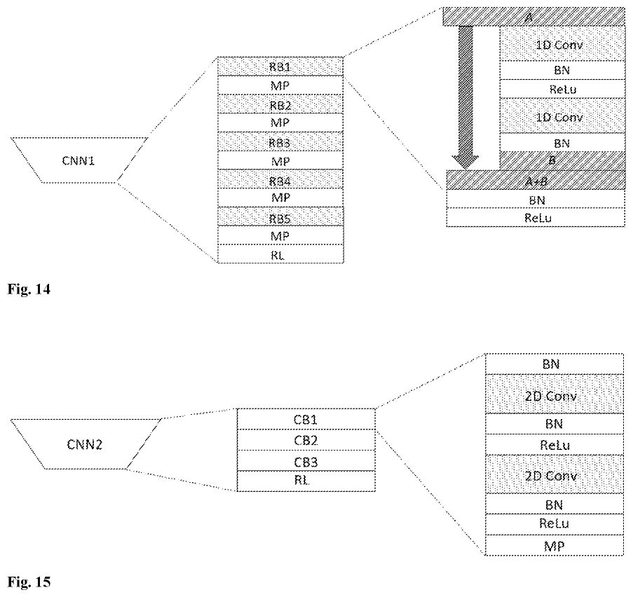

FIG. 14 shows schematically an example of the first convolutional neural network CNN1 of FIG. 13. The first convolutional neural network CNN1 comprises an alternating sequence of residual learning blocks (RB1, RB2, RB3, RB4, RB5) and max pooling layers (MP), followed by a reshape layer (RL). The reference A indicates the representation inputted into each residual block. Each residual learning block comprises a convolution layer (1D Conv), followed by a batch normalization layer (BN), followed by a rectified linear unit activation layer (ReLu), followed by a convolution layer (1D Conv), followed by a batch normalization layer (BN). The resulting representation B is then added to the original representation A, before the representation A+B is inputted into a batch normalization layer (BN), followed by a rectified linear unit activation layer (ReLu). For each representation A+B, the neural network learns to calculate B such that, when added to A, it reduces the loss values to a defined minimum (see e.g. K. He et al.: Deep Residual Learning for Image Recognition, 2015, arXiv:1512.03385v1 [cs.CV]).

FIG. 15 shows schematically an example of the second convolutional neural network CNN2 of FIG. 13. The second convolutional neural network CNN2 comprises three convolution blocks (CB1, CB2, CB3), and a reshape layer (RL). Each convolution block comprises a batch normalization layer BN, followed by a convolution layer (2D Conv) which performs 2D convolution operations, followed by a batch normalization layer (BN), followed by a rectified linear unit activation layer (ReLu), followed by a convolution layer (2D Conv) which performs 2D convolution operations, followed by a batch normalization layer (BN), followed by a rectified linear unit activation layer (ReLu), followed by a max pooling layer (MP).

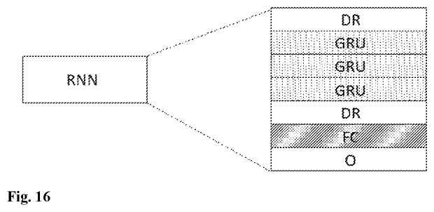

FIG. 16 shows schematically an example of the recurrent neural network RNN of FIG. 13. The recurrent neural network RNN comprises a dropout regularization (DR), followed by a three gated recurrent units (GRU), followed by a dropout regularization (DR), followed by fully connected layer(s) (FC), followed by an output layer (O).

“Batch normalization” is a normalization technique done between the layers of an artificial neural network. It is done along batches instead of the full data set. Batch normalization applies a transformation that maintains the mean output close to 0 and the output standard deviation close to 1. It serves to speed up training and use higher learning rates, making learning easier.

“Max pooling” is a discretization process. The objective is to down-sample an input representation, reducing its dimensionality and allowing for assumptions to be made about features contained in the sub-regions binned. Max Pooling is a pooling operation that calculates the maximum value for patches of a feature map and uses it to create a down-sampled (pooled) feature map.

As described above, “convolutional layers” systematically apply filters to input data in order to create feature maps that summarize the presence of those features in the input. Repeated application of the same filter to an input results in a map of activations called a feature map, indicating the locations and strength of a detected feature in an input, such as an image or a spectrogram.

The “rectified linear unit” is the most commonly used activation function in deep learning models. The function returns 0 if it receives any negative input, but for any positive value x it returns that value back.

A “reshape layer” is used to change the dimensions of its input, without changing its data.

“Dropout” is a regularization technique for reducing overfitting in artificial neural networks by preventing complex co-adaptations on training data. It is an efficient way of performing model averaging with neural networks. The term dropout refers to randomly dropping out, or omitting, units (both hidden and visible) during the training process of a neural network.

A “gated recurrent unit” is a type of recurrent neural network. It is similar to a long short-term memory (LSTM), but only has two gates, a reset gate and an update gate, and lacks an output gate.

The layers, units, functions, techniques and operations described above are described in more detail in various textbooks and publications (see e.g.: S. Kostadinov: Recurrent Neural Networks with Python, Quick Start Guide, Packt Publishing Ltd., 2018, ISBN: 978-1-78913-233-5; M. Sewak et al.: Practical Convolutional Neural Networks, Packt Publishing Ltd., 2018, ISBN: 978-1-78839-230-3; K. He et al.: Deep Residual Learning for Image Recognition, 2015, arXiv:1512.03385v1 [cs.CV]).

Further preferred embodiments of the present disclosure are:
1. A computer-implemented method, the method comprising the steps:
   receiving an audio recording
   generating a time-domain representation of the audio recording
   generating a spectrogram representation of the audio recording
   providing a trained machine learning model, wherein the trained machine learning model comprises a first input, a second input, and an output
   inputting the time-domain representation into the first input and the spectrogram representation into the second input, wherein the trained machine learning model is configured and trained to
      generate time-domain features on the basis of the time-domain representation,
      generate spectrogram features on the basis of the spectrogram representation,
      generate a joint representation on the basis of the time-domain features and the spectrogram features, and
      generate, on the basis of the joint representation, a cough information, the cough information indicating whether and/or to what extent the audio recording represents a cough
   receiving, from the trained machine learning model the cough information
   outputting the cough information.
2. The computer-implemented method according to embodiment 1, wherein the machine learning model comprises
   a first feature extraction unit which is configured to generate the time-domain features on the basis of the time-domain representation
   a second feature extraction unit which is configured to generate the spectrogram features on the basis of the spectrogram representation
   a feature combination unit which is configured to generate the joint representation on the basis of the time-domain features and the spectrogram features
   a cough detection and/or characterization unit which is configured to generate the cough information on the basis of the joint representation.
3. The computer-implemented method according to embodiment 2, wherein the first feature extraction unit and/or the second feature extraction unit is/are or comprises/comprise a convolutional neural network.
4. The computer-implemented method according to embodiment 3, wherein the convolutional neural network of the first feature extraction unit comprises shortcut connections.
5. The computer-implemented method according to any one of embodiments 2 to 4, wherein the cough detection and/or characterization unit is or comprises a recurrent neural network.
6. The computer-implemented method according to embodiment 5, wherein the recurrent neural network comprises gated recurrent units.
7. The computer-implemented method according to any one of embodiments 1 to 6, wherein the joint representation is generated from a time-domain feature vector representing the time-domain features and a spectrogram feature vector representing the spectrogram features by one or more of the following operations: element-wise multiplication, element-wise addition, cross-product, stacking on top or combinations thereof.
8. The computer-implemented method according to any one of embodiments 1 to 7, wherein the joint representation preserves the property of differentiability.
9. The computer-implemented method according to any one of embodiments 1 to 8, wherein the machine learning model outputs a vector, the vector containing a number of timesteps and for each time step a probability value, the probability value indicating the probability that the sound recorded at the respective timestep is caused by a cough.
10. The computer-implemented method according to any one of embodiments 1 to 9, wherein the machine learning model outputs a number, the number being equal to the number of coughs in the audio recording.
11. A computer system comprising:
a processor; and
a memory storing a computer program configured to perform, when executed by the processor, an operation, the operation comprising:
   receiving an audio recording
   generating a time-domain representation of the audio recording
   generating a spectrogram representation of the audio recording
   providing a trained machine learning model, wherein the trained machine learning model comprises a first input, a second input, and an output
   inputting the time-domain representation into the first input and the spectrogram representation into the second input, wherein the trained machine learning model is configured and trained to
      generate time-domain features on the basis of the time-domain representation,
      generate spectrogram features on the basis of the spectrogram representation,
      generate a joint representation on the basis of the time-domain features and the spectrogram features, and
      generate, on the basis of the joint representation, a cough information, the cough information indicating whether and/or to what extent the audio recording represents a cough
   receiving, from the trained machine learning model the cough information
   outputting the cough information.
12. A non-transitory computer readable medium having stored thereon software instructions that, when executed by a processor of a computer system, cause the computer system to execute the following steps:
   receiving an audio recording
   generating a time-domain representation of the audio recording
   generating a spectrogram representation of the audio recording
   providing a trained machine learning model, wherein the trained machine learning model comprises a first input, a second input, and an output
   inputting the time-domain representation into the first input and the spectrogram representation into the second input, wherein the trained machine learning model is configured and trained to
      generate time-domain features on the basis of the time-domain representation,
      generate spectrogram features on the basis of the spectrogram representation,
      generate a joint representation on the basis of the time-domain features and the spectrogram features, and
      generate, on the basis of the joint representation, a cough information, the cough information indicating whether and/or to what extent the audio recording represents a cough
   receiving, from the trained machine learning model the cough information
   outputting the cough information.

The operations in accordance with the teachings herein may be performed by at least one computer system specially constructed for the desired purposes or at least one general-purpose computer system specially configured for the desired purpose by at least one computer program stored in a typically non-transitory computer readable storage medium.

The term “non-transitory” is used herein to exclude transitory, propagating signals or waves, but to otherwise include any volatile or non-volatile computer memory technology suitable to the application.

A “computer system” is a system for electronic data processing that processes data by means of programmable calculation rules. Such a system usually comprises a “computer”, that unit which comprises a processor for carrying out logical operations, and also peripherals.

In computer technology, “peripherals” refer to all devices which are connected to the computer and serve for the control of the computer and/or as input and output devices. Examples thereof are monitor (screen), printer, scanner, mouse, keyboard, drives, camera, microphone, loudspeaker, etc. Internal ports and expansion cards are, too, considered to be peripherals in computer technology.

Computer systems of today are frequently divided into desktop PCs, portable PCs, laptops, notebooks, netbooks and tablet PCs and so-called handhelds (e.g. smartphone); all these systems can be utilized for carrying out the invention.

The term “process” as used above is intended to include any type of computation or manipulation or transformation of data represented as physical, e.g. electronic, phenomena which may occur or reside e.g. within registers and/or memories of at least one computer or processor. The term processor includes a single processing unit or a plurality of distributed or remote such units.

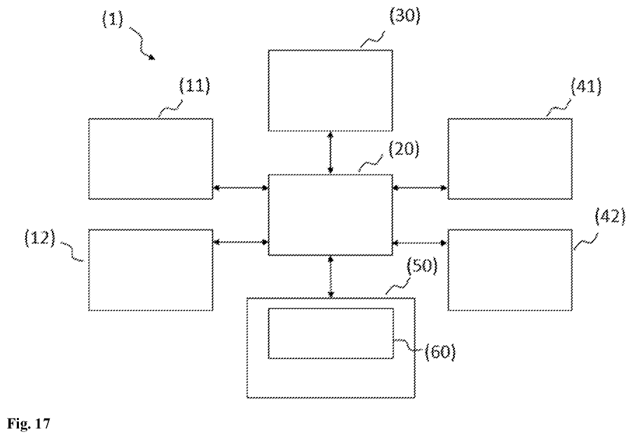

FIG. 17 illustrates a computer system (1) according to some example implementations of the present disclosure in more detail.

Generally, a computer system of exemplary implementations of the present disclosure may be referred to as a computer and may comprise, include, or be embodied in one or more fixed or portable electronic devices. The computer may include one or more of each of a number of components such as, for example, a processing unit (20) connected to a memory (50) (e.g., storage device).

The processing unit (20) may be composed of one or more processors alone or in combination with one or more memories. The processing unit (20) is generally any piece of computer hardware that is capable of processing information such as, for example, data, computer programs and/or other suitable electronic information. The processing unit (20) is composed of a collection of electronic circuits some of which may be packaged as an integrated circuit or multiple interconnected integrated circuits (an integrated circuit at times more commonly referred to as a “chip”). The processing unit (20) may be configured to execute computer programs, which may be stored onboard the processing unit (20) or otherwise stored in the memory (50) of the same or another computer.

The processing unit (20) may be a number of processors, a multi-core processor or some other type of processor, depending on the particular implementation. For example, it may be a central processing unit (CPU), a field programmable gate array (FPGA), a graphics processing unit (GPU) and/or a tensor processing unit (TPU). Further, the processing unit (20) may be implemented using a number of heterogeneous processor systems in which a main processor is present with one or more secondary processors on a single chip. As another illustrative example, the processing unit (20) may be a symmetric multi-processor system containing multiple processors of the same type. In yet another example, the processing unit (20) may be embodied as or otherwise include one or more ASICs, FPGAs or the like. Thus, although the processing unit (20) may be capable of executing a computer program to perform one or more functions, the processing unit (20) of various examples may be capable of performing one or more functions without the aid of a computer program. In either instance, the processing unit (20) may be appropriately programmed to perform functions or operations according to example implementations of the present disclosure.

The memory (50) is generally any piece of computer hardware that is capable of storing information such as, for example, data, computer programs (e.g., computer-readable program code (60)) and/or other suitable information either on a temporary basis and/or a permanent basis. The memory (50) may include volatile and/or non-volatile memory, and may be fixed or removable. Examples of suitable memory include random access memory (RAM), read-only memory (ROM), a hard drive, a flash memory, a thumb drive, a removable computer diskette, an optical disk, a magnetic tape or some combination of the above. Optical disks may include compact disk-read only memory (CD-ROM), compact disk-read/write (CD-R/W), DVD, Blu-ray disk or the like. In various instances, the memory may be referred to as a computer-readable storage medium. The computer-readable storage medium is a non-transitory device capable of storing information, and is distinguishable from computer-readable transmission media such as electronic transitory signals capable of carrying information from one location to another. Computer-readable medium as described herein may generally refer to a computer-readable storage medium or computer-readable transmission medium.

The machine learning model, the trained machine learning model and the segmentation unit may be stored in the memory (50).

In addition to the memory (50), the processing unit (20) may also be connected to one or more interfaces for displaying, transmitting and/or receiving information. The interfaces may include one or more communications interfaces and/or one or more user interfaces. The communications interface(s) may be configured to transmit and/or receive information, such as to and/or from other computer(s), network(s), database(s) or the like. The communications interface may be configured to transmit and/or receive information by physical (wired) and/or wireless communications links. The communications interface(s) may include interface(s) (41) to connect to a network, such as using technologies such as cellular telephone, Wi-Fi, satellite, cable, digital subscriber line (DSL), fiber optics and the like. In some examples, the communications interface(s) may include one or more short-range communications interfaces (42) configured to connect devices using short-range communications technologies such as NFC, RFID, Bluetooth, Bluetooth LE, ZigBee, infrared (e.g., IrDA) or the like.

The user interfaces may include a display (30). The display may be configured to present or otherwise display information to a user, suitable examples of which include a liquid crystal display (LCD), light-emitting diode display (LED), plasma display panel (PDP) or the like. The user input interface(s) (11) may be wired or wireless, and may be configured to receive information from a user into the computer system (1), such as for processing, storage and/or display. Suitable examples of user input interfaces include a microphone, image or video capture device, keyboard or keypad, joystick, touch-sensitive surface (separate from or integrated into a touchscreen) or the like. In some examples, the user interfaces may include automatic identification and data capture (AIDC) technology (12) for machine-readable information. This may include barcode, radio frequency identification (RFID), magnetic stripes, optical character recognition (OCR), integrated circuit card (ICC), and the like. The user interfaces may further include one or more interfaces for communicating with peripherals such as printers and the like.

As indicated above, program code instructions (60) may be stored in memory (50), and executed by processing unit (20) that is thereby programmed, to implement functions of the systems, subsystems, tools and their respective elements described herein. As will be appreciated, any suitable program code instructions (60) may be loaded onto a computer or other programmable apparatus from a computer-readable storage medium to produce a particular machine, such that the particular machine becomes a means for implementing the functions specified herein. These program code instructions (60) may also be stored in a computer-readable storage medium that can direct a computer, processing unit or other programmable apparatus to function in a particular manner to thereby generate a particular machine or particular article of manufacture. The instructions stored in the computer-readable storage medium may produce an article of manufacture, where the article of manufacture becomes a means for implementing functions described herein. The program code instructions (60) may be retrieved from a computer-readable storage medium and loaded into a computer, processing unit or other programmable apparatus to configure the computer, processing unit or other programmable apparatus to execute operations to be performed on or by the computer, processing unit or other programmable apparatus.

Retrieval, loading and execution of the program code instructions (60) may be performed sequentially such that one instruction is retrieved, loaded and executed at a time. In some example implementations, retrieval, loading and/or execution may be performed in parallel such that multiple instructions are retrieved, loaded, and/or executed together. Execution of the program code instructions (60) may produce a computer-implemented process such that the instructions executed by the computer, processing circuitry or other programmable apparatus provide operations for implementing functions described herein.

Execution of instructions by processing unit, or storage of instructions in a computer-readable storage medium, supports combinations of operations for performing the specified functions. In this manner, a computer system (1) may include processing unit (20) and a computer-readable storage medium or memory (50) coupled to the processing circuitry, where the processing circuitry is configured to execute computer-readable program code instructions (60) stored in the memory (50). It will also be understood that one or more functions, and combinations of functions, may be implemented by special purpose hardware-based computer systems and/or processing circuitry which perform the specified functions, or combinations of special purpose hardware and program code instructions.

### Example

The training data comprised full day recordings from 27 subjects, around 50000 single cough recordings from various people and around 100 h various non-cough background and disturbance sounds. A total of around 400 hours of recording material was available.

In a preprocessing, the audio recordings were divided into 5-second sections (window stride 3 seconds). The resolution of each section was 16 kHz, 32 bit float. So, each section comprised 16 kHz·5s=80,000 timesteps. For each audio recording information (target information) was available whether a cough is present in the audio recording and at which time interval the cough is present (determined by human experts).

Each 5-second section was transformed into a spectrogram representation using short-time Fourier transformation (window width 512, window stride 64), resulting in 1250 timesteps, each consisting of 64 frequency bins.

Each 5-second section was down-sampled to 8 kHz and normalized to form the time-domain representation, consisting of 40,000 timesteps each consisting of an amplitude between −1 and +1.

Each pair of corresponding spectrogram representation and time-domain representation was inputted into a machine learning model. “Corresponding” means that the spectrogram representation and the time-domain representation originate from the same audio recording section.

The machine learning model used for training and making prediction was the model as described herein in connection with FIGS. 13 to 16.

For the 2D convolution layers, the following parameters were chosen: filters 16, kernel (3, 3), stride (1, 1). For the 1D convolution layers, the following parameters were chosen: filters 64, kernel 3, stride 1.

For the 1D max pooling operations in CNN1, the following strides were chosen (top down): 4, 4, 4, 2, 2. For the 2D max pooling operations in CNN2, a stride of 2 was chosen.

For each time-domain representation, CNN1 generated a time-domain feature vector containing 157 timesteps and 64 information channels.

For each time-domain representation, CNN2 generated a spectrogram feature vector containing 157 timesteps and 128 information channels.

Since the time-domain feature vector and the spectrogram feature vector have the same number of timesteps, the two matrices were simply stacked on top of each other, resulting in a single matrix containing 157 timesteps and 192 information channels (the joint representation).

The joint representation was processed by three gated recurrent unit (GRU) layers before being channeled by a fully connected layer to get the output. Each GRU layer is bi-directional, comprises 64 units and returns a sequence. The fully connected layer comprises a sigmoid activation. A dropout rate of 0.5 was chosen in the RNN.

The output contained 157 timesteps (1 timestep=5 s/157=31.85 ms) each timestep contained a single number in the range from 0 to 1. This number represents the probability that the sound recorded at the respective timestep is caused by cough (1) or not (0). For example, the number 0.8 means that the probability that the sound recorded at the respective timestep is caused by cough is 80%.

The machine learning model was trained end-to-end by backpropagation using a cross-entropy loss function.

For comparison purposes, two additional machine learning models were trained. One model was trained only on time-domain representations (and the respective target information), the other was trained only on spectrogram representations (and the respective target information).

The following Table 1 shows a comparison of predictions made (i) only on the basis of time-domain representations, (ii) only on the basis of spectrogram representations, and (iii) on the basis of time-domain representations and spectrogram representations as described herein. The results summarized in Table 1 clearly demonstrate the superiority of the hybrid-model (time-domain representation+spectrogram representation) over the individual models.

TABLE 1

| Representation | Precision | Recall | AUC |
|---|---|---|---|
| (i) waveform | 81.03% ± 9.12% | 83.32% ± 13.47% | 0.877 |
| (ii) spectrogram | 93.97% ± 5.77% | 82.73% ± 14.46% | 0.912 |
| (iii) hybrid | 89.66% ± 6.78% | 90.81% ± 9.06% | 0.944 |
| (waveform + spectrogram) | | | |

1. A computer-implemented method, the method comprising the steps:
receiving one or more signals of one or more modalities, the signal(s) being related to an event,
providing a trained machine learning model, wherein the trained machine learning model comprises an output, and, for each signal, a first input, and a second input,
for each signal:
generating a time-domain representation of the signal,
generating a spectrogram representation of the signal,
inputting the time-domain representation into the first input of the trained machine learning model and the spectrogram representation into the second input of the machine learning model, wherein the trained machine learning model is configured and trained to
generate time-domain features on the basis of the time-domain representation,
generate spectrogram features on the basis of the spectrogram representation,
generate a joint representation on the basis of the time-domain features and the spectrogram features of all signals, and
generate, on the basis of the joint representation, an event information, the event information indicating whether and/or to what extent the event is related to a body action,
receiving, from the trained machine learning model the event information,
outputting the event information.

2. The computer-implemented method according to claim 1, wherein the body action is selected from: cough, snoring, sneezing, hiccups, vomiting, shouting, swallowing, wheezing, shortness of breath, chewing, teeth grinding, chills, convulsions, spasm.

3. The computer-implemented method according to claim 1 or 2, wherein the body action is or comprises one or more cough events.

4. The computer-implemented method according to any one of claims 1 to 3, wherein at least one signal of the one or more signals is an audio signal.

5. The computer-implemented method according to any one of claims 1 to 4, wherein at least one signal of the one or more signals is selected from: an electromyographic signal, an electrocardiogram signal, an accelerometer signal, a chest impedance signal, a plethysmographic signal, a temperature signal, a heart rate signal, a blood pressure signal.

6. The computer-implemented method according to any one of claims 1 to 5,
wherein the trained machine learning model comprises
a number n of first inputs,
a number n of second inputs,
a number n of first feature extraction units,
a number n of second feature extraction units,
one or more feature combination units,
a body action detection, identification and/or characterization unit, and
an output,
wherein n is the number of signals, wherein n is an integer equal to or greater than 1,
wherein each first input is configured to receive a time-domain representation of a signal,
wherein each second input is configured to receive a spectrogram representation of a signal,
wherein each first feature extraction unit is configured to generate, on the basis a time-domain representation, time-domain features,
wherein each second feature extraction unit is configured to generate, on the basis a spectrogram representation, spectrogram features,
wherein the one or more feature combination units are configured to generate, at least partially on all time-domain features and all spectrogram features, the joint representation,
wherein the body action detection, identification and/or characterization unit is configured to generate, on the basis of the joint representation, the event information,
wherein the output is configured to output the event information.

7. The computer-implemented method according to claim 6, wherein one or more of the first feature extraction units and/or the second feature extraction units are or comprise a convolutional neural network.

8. The computer-implemented method according to claim 7, wherein the convolutional neural network of one or more of the first feature extraction units comprise shortcut connections.

9. The computer-implemented method according to any one of claims 6 to 8, wherein the body action detection, identification and/or characterization unit is or comprises a recurrent neural network.

10. The computer-implemented method according to claim 9, wherein the recurrent neural network comprises gated recurrent units.

11. The computer-implemented method according to any one or claims 1 to 10, wherein the joint representation is generated, at least partially on the basis of one or more time-domain feature vectors representing one or more time-domain features and one or more spectrogram feature vectors representing one or more spectrogram features by one or more of the following operations: element-wise multiplication, element-wise addition, cross-product, stacking on top or combinations thereof.

12. The computer-implemented method according to any one or claims 1 to 11, wherein the joint representation preserves the property of differentiability.

13. The computer-implemented method according to any one or claims 1 to 12, wherein the trained machine learning model is configured to output a vector, the vector containing a number of timesteps and for each time step a probability value, the probability value indicating the probability that the one or more signals at the respective timestep are caused by a cough event.

14. The computer-implemented method according to any one or claims 1 to 13, wherein the trained machine learning model is configured to output a number, the number being equal to the number of cough events captured in the one or more signals.

15. A computer system comprising:
a processor; and
a memory storing an application program configured to perform, when executed by the processor, an operation, the operation comprising:
receiving one or more signals of one or more modalities, the signal(s) being related to an event,
providing a trained machine learning model, wherein the trained machine learning model comprises an output, and, for each signal, a first input, and a second input,
for each signal:
generating a time-domain representation of the signal,
generating a spectrogram representation of the signal,
inputting the time-domain representation into the first input of the trained machine learning model and the spectrogram representation into the second input of the machine learning model, wherein the trained machine learning model is configured and trained to
generate time-domain features on the basis of the time-domain representation,
generate spectrogram features on the basis of the spectrogram representation,
generate a joint representation on the basis of the time-domain features and the spectrogram features of all signals, and
generate, on the basis of the joint representation, an event information, the event information indicating whether and/or to what extent the event is related to a body action,
receiving, from the trained machine learning model the event information,
outputting the event information.

16. A non-transitory computer readable medium having stored thereon software instructions that, when executed by a processor of a computer system, cause the computer system to execute the following steps:
receiving one or more signals of one or more modalities, the signal(s) being related to an event,
providing a trained machine learning model, wherein the trained machine learning model comprises an output, and, for each signal, a first input, and a second input,
for each signal:
generating a time-domain representation of the signal,
generating a spectrogram representation of the signal,
inputting the time-domain representation into the first input of the trained machine learning model and the spectrogram representation into the second input of the machine learning model, wherein the trained machine learning model is configured and trained to
generate time-domain features on the basis of the time-domain representation,
generate spectrogram features on the basis of the spectrogram representation,
generate a joint representation on the basis of the time-domain features and the spectrogram features of all signals, and
generate, on the basis of the joint representation, an event information, the event information indicating whether and/or to what extent the event is related to a body action,
receiving, from the trained machine learning model the event information,
outputting the event information.
